(19) United States
(12) Patent Application Publication
(10) Pub. No.: US 2005/0249790 A1
(43) Pub. Date: Nov. 10, 2005

(54) Polymerized hydrogel adhesives with high levels of monomer units in salt form

(75) Inventors: Christian Hubert Weidl, Mannheim (DE); Felix Christian Gorth, Ludwigshafen (DE); Volker Frenz, Mainz-Kostheim (DE)

Correspondence Address: MARSHALL, GERSTEIN & BORUN LLP, 233 S. WACKER DRIVE, SUITE 6300, SEARS TOWER, CHICAGO, IL, 60606, US

(73) Assignee: BASF Aktiengesellschaft, Ludwigshafen (DE)

(21) Appl. No.: 10/528,302

(22) PCT Filed: Sep. 18, 2003
(86) PCT No.: PCT/EP03/10380

§ 371 Date: Mar. 17, 2005

(30) Foreign Application Priority Data
Sep. 27, 2002 (DE) 02021629.7

(51) Int. Cl. A61F 13/00

(52) U.S. Cl. 424/443

(57) ABSTRACT

The present invention relates to hydrogel adhesives which are capable of attaching to mammalian skin, exhibit excellent attachment and painless removal properties, and which show excellent stability upon storage at room temperature or even at elevated temperatures and for longer periods of time. The adhesive hydrogel described in the present invention can be used as body adhesive in products for e.g. personal care, medical devices, beauty care and a variety of functional articles to be worn by a human. The hydrogel adhesive of the invention comprises 10-60 wt. % of a cross-linked hydrophilic polymer, 5-80 wt. % of a water-soluble non-ionic humectant, and from about 10-85 wt. % water, wherein the hydrophilic polymer comprises at least 80 mole % of one or more weak-acid monomer units having a pKa above 3, the weak-acid monomer being more than 60 mole % in its salt form, the level of monomer in acid form in said hydrophilic polymer not exceeding 50 mole % of all monomer units and the hydrogel adhesive having a peel strength on PET of 0.3 to 5.0 N/cm and a stability index measured after 14 days SIx 14 below 0.50, preferably below 0.20, most preferably below 0.10.

The present invention relates to hydrogel adhesives which are capable of attaching to mammalian skin, exhibit excellent attachment and painless removal properties, and which show excellent stability upon storage at room temperature or even at elevated temperatures and for longer periods of time. The adhesive hydrogel described in the present invention can be used as body adhesive in products for e.g. personal care, medical devices, beauty care and a variety of functional articles to be worn by a human.

While hydrogel body adhesives for use in consumer products such as absorbent articles and waste-management articles have previously been described in, respectively, EP 1025823 and EP 1025866, the disclosure of hydrogel-adhesive has mainly occurred in the context of medical applications, such as skin electrodes, transdermal drug delivery and wound healing. In EP 1025823 and EP 1025866, certain needs for consumer products such as absorbent and human waste-management products are disclosed, including secure attachment, painless removal and stability of adhesion in presence of excess moisture. In WO 00/46319 and WO 00/45864 are disclosed hydrogel adhesives for use in e.g. biomedical skin showing improved adhesion on wet skin and oily skin.

The hydrogel adhesives are prepared by polymerization of aqueous reaction mixtures comprising at least one ionic water-soluble monomer and at least one non-ionic water-soluble monomer.

The examples of the above mentioned applications disclosed the use of sodium-2-acrylamido-2-methylpropanesulfonate (NaAMPS) as ionic monomer and N,N-dimethylacrylamide (NNDMA) as non-ionic monomer.

In WO 00/45698, U.S. Pat. No. 4,848,353, U.S. Pat. No. 4,539,996 and WO 97/24378 are disclosed pressure-sensitive adhesives for electrodes.

WO 00/45698 and U.S. Pat. No. 4,848,353 recommend the co-polymerization of acrylic acid and N-vinypyrrolidone to get hydrogels with improved adhesive properties.

U.S. Pat. No. 4,539,996 discloses conductive adhesives based on acrylic acid. The degrees of neutralization of the conductive adhesives are low.

WO 97/24378 discloses hydrophilic pressure sensitive adhesive compositions. The hydrophilic adhesives are prepared by crosslinking of non-crosslinked polyacrylic acid.

It is critical for these hydrogel body adhesives that they show an excellent long term stability to storage and transportation conditions at room temperature or even at elevated temperatures. Otherwise the products containing said hydrogels will not have a sufficient shelf life to satisfy consumer needs.

In U.S. Pat. No. 5,665,477 a biocompatible hydrogel adhesive is disclosed. The use of alkanolamines such as diisopropanolamine provides good wet tack properties of the body adhesives. But the hydrogel adhesives discolour and get an unpleasant odour on longer shelf times due to the alkanolamine content.

An object of the present invention is to provide hydrogel body adhesives with an improved shelf life.

Another object of the present invention is to provide hydrogel body adhesives with reduced discolouration and without liberation of unpleasant odours on longer shelf times.

It has now been found that hydrogel compositions showing excellent storage stability, can be formulated through the selection of the level of monomers units in salt form in said compositions.

The present invention relates to hydrogel adhesives and their use for attachment to mammalian skin comprising 10-60 wt % of a crosslinked hydrophilic polymer; 5-80 wt % of a water-soluble nonionic humectant, and from about 10-85 wt % water wherein the hydrophilic polymer comprises at least 50 mole %, preferably 80 mole %, more preferably 90 mole %, most preferably 95 mole % or even 100% of one or more weak-acid monomer units having a pKa above 3, the weak-acid monomer being more than 50 mole %, preferably at least 55 mole %, more preferably at least 60 mole %, most preferably in the range of 60 mole % to 80 mole % in its salt form, the level of monomer in acid form in said hydrophilic polymer not exceeding 50 mole % of all monomer units, and the hydrogel adhesive having a peel strength on PET of 0.3 to 5.0 N/cm. The peel strength on PET of 0.3 to 5.0 should be measured on the day x.

The nonionic humectant is preferably glycerol, and the weak-acid is preferably acrylic acid.

The hydrogel adhesives described in this invention show an excellent long term storage stability at room temperature or even at elevated temperatures, meaning they do not harden, characterized by having a stability index SIₓ₁₄ smaller than 0.50, preferably smaller than 0.20, most preferably smaller than 0.10. The hydrogel adhesives have on the day x the above mentioned peel strength on PET of 0.3 to 5.0.

The yellowing of the hydrogel adhesives of the invention after 14 days of the rapid ageing test is less than 80, preferred less than 70, and most preferred less than 60.

The hydrogel adhesives of the invention are waterstable, i.e. they do not degrade to a substantial amount in water. Preferable less than 20 wt. % of the polymer, more preferably less than 15 wt. %, most preferably less than 10 wt. % are solvable in water.

The hydrogel adhesives herein contain 10-60 wt % of a cross-linked hydrophilic polymer, 5-80 wt % of a water-soluble nonionic humectant, and 10-85 wt % water. The polymerization of the monomers preferably takes place in presence of the nonionic humectant and water and cross-linking creates a 3-dimensional matrix for the polymer, also referred to as gel form and hydrogel. In general, the hydrogel adhesive consists of one or only a few (less than 100) 3 dimensional matrices. Each 3-dimensional matrix shows normally geometrical dimensions in the range of at least 5 mm, preferred of at least 1 cm. In general, the 3-dimensional matrix consists only of one homogeneous phase.

The hydrophilic polymer includes repeating units or monomers which contain at least 50 mole % of one or more weak-acid monomers, more preferably more than 80 mole %, most preferably 100 mole % of said weak-acid monomers.

The weak-acid monomer being more than 50 mole %, preferably at least 55 mole %, more preferably at least 60 mole %, most preferably in the range of 60 mole % to 80 mole % in its salt form, the level of monomer in acid form in said hydrophilic polymer not exceeding 50 mole % of all monomer units.

The hydrogel adhesives have preferably a pH-value of 5.0 to 8.0, more preferably of 5.2 to 6.0.

Weak-Acid Monomer:

The weak acid monomer is defined in relation to its pKa, which must be above 3. The said monomers are preferably selected from the group of olefinically unsaturated carboxylic acids and carboxylic acid anhydrides such as acrylic acid, methacyclic acid, maleic acid, itaconic acid, crotonic acid, ethacrylic acid, citroconic acid, fumaric acid, â-sterylacrylic acid and the like. Particularly preferred weak-acid monomers are acrylic acid and methacrylic acid, acrylic acid being most preferred.

Humectant:

The 3-dimensional adhesive matrix also comprises a humectant or mixture of humectants (also referred herein as a plasticizer), which is preferably a liquid at room temperature. The humectant is selected such that the monomer and polymer may be solubilized or dispersed within. For embodiments wherein irradiation cross linking is to be carried out, the humectant is desiderably irradiation cross linking compatible such that is does not significantly inhibit the irradiation cross linking process of the polymer. The components of the humectant mixture are preferably hydrophilic and miscible with water.

Suitable humectants include alcohols, polyhydric alcohols such as glycerol and sorbitol, and glycols and ether glycol such as mono- or diethers of polyalkylene glycol, mono- or diester polyalkylene glycols, polyethylene glycols (typically up to a molecular weight of about 600), glycolates, glycerol, sorbitan esters, esters of citric and tartaric acid, imidazoline derived amphoteric surfactants, lactams, amides, polyamides, quaternary ammonium compounds, esters such as phthalates, adipates, stearates, palmitates, sebacates, or myristates, glycerol esters, including mono/di/tri-glycerides, and combinations thereof. Particularly preferred are polyhydric alcohols, polyethylene glycol (with a molecular weight up to about 600), glycerol, sorbitol and mixtures thereof. Glycerol is especially preferred. The humectant comprises 5-80 wt % of the hydrogel.

An important function of the humectant is to reduce the water activity of the hydrogel to 0.35-0.95, preferably 0.4-0.85, more preferably from 0.45-0.75, most preferably 0.5-0.65. Water activity is determined by measuring the equilibrium relative humidity above the hydrogel according to the method described hereinafter in the test methods section.

Rheology:

The viscous behaviour of the adhesive can be interpreted to represent an indication of the ability of the adhesive to quickly attach and securely adhere to a particular surface. The elastic behaviour can be interpreted as an indication of the “hardness” behaviour of the adhesive. Its value is also important for good initial attachment. Their combination is believed to be an indicator of the required force upon removal. The relation between elastic and viscous modulus is considered to be an indication on which fraction of the removal energy will be dissipated within the adhesive and which fraction is available to trigger the actual removal.

In order to provide adhesives for secure initial and prolonged attachment and easy/painless removal, the relation between the elastic modulus and the viscous modulus as well as their dynamic behaviour is also of importance. While not being bound by theory, it is believed that for hydrogels applied to skin, the rheological properties at T=37° C. are most relevant to adhesion and removal properties. However, for the hydrogels of this invention, it has been found that the rheology properties are only at most moderately sensitive to temperature in the range of 25-37° C. Thus, for the purpose of this invention, it is convenient to specify the rheological properties at a temperature of 25° C. The adhesive has an elastic modulus at a temperature of 25° C. abbreviated G′₂₅, a viscous modulus at a temperature of 25° C. of G″25, and the ratio of G″₂₅/G′₂₅ at 25° C., referred to as tan δ₂₅.

It has been found that, in order to perform effectively the adhesives according to the present invention must have a G′₂₅ (1 rad/sec) in the range 100-20000 Pa, preferably in the range between 1000 and 10000 Pa, most preferably in the range of 2000 to 6000 Pa.

It is also an important attribute to the composition, herein that they exhibit very good cohesiveness, to prevent residue of adhesive on the skin.

Stability Index:

The stability index describes the resistance of the Hydrogel adhesive against storage and/or transportation conditions. These conditions are e.g. room temperature or elevated temperatures. To simulate the storage or transportation conditions a rapid ageing test was used as described in the test method section. The effect of ageing is increasing with time and can already be seen clearly after 14 days. For this a stability index after 14 days (SI₁₄) is defined as follows:
SI₁₄=abs(1−(G′₂₅¹⁴/G′₂₅⁰))
with G′₂₅⁰ being the initial G′₂₅ (1 rad/sec) value of the fresh product and G′₂₅¹⁴ being the G′₂₅ (1 rad/sec) value of the hydrogel after 14 days of the rapid ageing test.

In addition to the SI₁₄ a general stability index SIₓ₁₄ can be defined as follows:
SIₓ₁₄=abs(1−(G′₂₅^{14+x}/G′₂₅^{x}))

The SIₓ₁₄ takes into account the aging properties of an x days old Hydrogel after an 14 days (at day x+14) stability test. The Hydrogels of the invention have preferably an initial G′₂₅ (1 rad/sec) value at the day 0 or the day x of between 100 and 20000 Pa, preferably between 1000 and 10000 Pa and more preferably between 2000 and 6000 Pa and/or a peel strength on PET of 0.3 to 5.0 N/cm, preferably between 0.5 to 3.0 N/cm and more preferably between 0.8 to 2.0 N/cm.

In analogy to the SI₁₄ and SIₓ₁₄ other stability indexes are imaginable for different storage times, such as SI₇ or SI₂₈ for a 7 or 28 day storage time.

The measurements for the determination of the SI indexes should be performed 12 months, preferably 6 months, more preferably 3 months after production of the hydrogel.

The Hydrogels described in this invention show a stability index SI₁₄ of less than 0.5, preferably less than 0.2, most preferably less than 0.1.

The Hydrogels described in this invention show a stability index SI^{x14} of less than 0.5, preferably less than 0.2, most preferably less than 0.1. Preferred are Hydrogels showing a SI₁₄ and a SIₓ₁₄ value in the above mentioned ranges.

Adhesion Properties:

The hydrogels herein preferably have a 90° peel force on dry skin of between 0.3 to 5 N/cm, more preferably 1.5 to 3 N/cm. Peel force can also be measured at 180° on Polyethyleneterephthalate (PET). The hydrogels herein preferably have a peel force on PET of between 0.3 to 5.0 N/cm, preferably between 0.5 to 3.0 N/cm and more preferably between 0.8 to 2.0 N/cm. The methods for measuring peel force on skin and PET are described hereinafter in the test methods section.

Preferred Hydrogels

Preferred hydrogels according to a specific embodiment of the present invention combine a peel force as given above, with an excellent stability to storage and transportation at room temperature or even at elevated temperatures, characterized by the stability index SI₁₄ and/or SIₓ₁₄ for each specific G′₂₅ (1 rad/sec) of less than 0.5, preferably less than 0.2, most preferably less than 0.1.

It has been found that the maintenance of both characteristics in said ranges is warranted if the level of weak-acid preferably acrylic acid in the hydrogels herein, is at least 90 mole %, preferably at least 95 mole % and said weak-acid is more than 50 mole %, preferably 55 mole %, more preferably at least 60 mole % in its salt form, more preferably 60 mole % to 80 mole % in its salt form.

Accordingly such preferred hydrogels of the present invention for attachment to mammalian skin comprise 10-60 wt % of a cross-linked hydrophilic polymer, 5-80 wt % of a water-soluble non-ionic humectant and 10-85 wt % water, characterized in that the polymer comprises at least 90 mole % weak-acid monomer, preferably 100 mole % weak-acid monomer, where the weak-acid monomer is preferably acrylic acid, where the weak-acid monomer is more than 50 mole % in its salt form, preferably at least 55 mole %, more preferably at least 60 mole % in its salt form, more preferably 60 mole % to 80 mole %, and wherein G′₂₅ (1 rad/sec) is in the range of 100 Pa to 20000 Pa, preferably in the range between 1000 and 10000 Pa, most preferably 2,000-6000 Pa, the humectant being preferably glycerol.

Said hydrogels according to the embodiment herein, are preferably such that the counterion for the weak-acid monomer unit in salt form is a mono, di, or tri-valent metal ion or combination thereof. Sodium and potassium are especially preferred counterions.

Polymerization Conditions:

According to the present invention the polymer component of the adhesive can be physically, chemically or ionically cross linked in order to form the 3 dimensional matrix. Physical cross linking refers to polymers having cross links which are not chemical covalent bonds but are of a physical nature such that for example there are areas in the 3 dimensional matrix having high crystallinity or areas having a high glass transition temperature or areas having hydrophobic interactions. Chemical cross linking refers to polymers which are linked by covalent chemical bonds. The polymer can be chemically cross linked by radiation techniques such as UV-, E-beam-, gamma or micro-wave radiation or, preferably by co-polymerizing the monomers with a di/poly-functional monomer crosslinker via the use e.g., of UV, thermal and/or redox polymerization initiators.

Suitable polyfunctional monomers, monomer crosslinkers include polyethyleneoxide di(meth)acrylates with varying PEG molecular weights, IRR280 (a PEG diacrylate available from UCB Chemical), trimethylolpropane ethoxylate tri(meth)acrylate with varying ethyleneoxide molecular weights, IRR210 (an alkoxylated triacrylate: available from UCB Chemicals), trimethylolpropane tri(meth)acrylate, divinylbenzene, pentaerythritol triacrylate, pentaerythritol triallyl ether, triallyl amine, N,N-methylene-bis-acrylamide and other polyfunctional monomer crosslinkers known to the art. Preferred polyfunctional monomer crosslinkers include the polyfunctional diacrylates and triacrylates.

The monomers of the present invention are preferably polymerized via the use of a free radical polymerization initiatior. Such free-radical polymerization initiators are well known in the art and can be one or more photoinitiator(s), thermal initiator(s), or redox initiator(s) and be present in quantities up to 5% by weight, preferably from 0.02% to 2%, more preferably from 0.02% to 0.4%. Photoinitiators are preferred. Suitable photoinitiators include type 1-hydroxy-ketones and benzyldimethyl-ketals e.g. Irgacure 651 (dimethoxybenzylphenone; available from Ciba Specialty Chemicals) which are believed, on irradiation with UV frequencies, to form benzoyl radicals that initiate polymerization. Particularly preferred photoinitiators include 2-hydroxy-2-methyl-propiophenone (available under the trade name of Darocur 1173 from Ciba Specialty Chemicals), 1-hydroxycyclohe-xylphenylketone (available under the trade name Irgacure 184 from Ciba Specialty Chemicals) and 4-(2-hydroxy-ethoxy)phenyl-(2-hydroxy-2-methylpropyl) ketone (available under the trade name of Irgacure 2959 from Ciba Specialty Chemicals). Suitable thermal initiators include potassium persulfate, V50 and VA044 (available from Wako). Suitable redox initiators include the combination of hydrogen peroxide and ascorbic acid, sodium persulfate and ascorbic acid or Fe(II) and hydrogen peroxide.

Chemical crosslinking can also be effected after polymerization by use of polyfunctional reagents capable of reacting with polymer functional groups such as ethyleneglycol diglycidyl ether, polyols such as glycerol, diepoxides such as Denacol EX 810, and other polyfunctional reagents known to the art.

Crosslinking can also be effected all or in part by ionic crosslinking wherein groups of opposite charge interact via ionic interactions. Suitable ionic crosslinking agents include those known to the art including polyvalent cations such as Al³⁺, Fe³⁺, Ca²⁺ and Mg²⁺, di/poly-amines, di/poly-quaternary ammonium compounds, including polymeric polyamines and polyquaternary ammonium compounds known to the art.

In preparing adhesive compositions in accordance with the invention, the ingredients will usually be mixed to provide a reaction mixture in the form of an initial pre-gel aqueous based liquid formulation, and this is then converted into a gel by a free radical polymerization reaction as described above. This may be achieved for example using conventional thermal initiators and/or photoinitiators or by ionizing radiation. Photoinitiation is a preferred method and will usually be applied by subjecting the pre-gel reaction mixture containing an appropriate photo-initiation agent to UV light after it has been spread or coated as a layer on siliconised release paper or other solid or porous substrate. The incident UV intensity, at a wavelength in the range from 240 to 420 nm is of sufficient intensity and exposure duration (e.g. 10-3000 mW/cm²) to complete the polymerization in a reasonable time. To facilitate the process, it is often preferable to expose the reaction mixture to several UV irradiation sources, in sequence. The processing will generally be carried out in a controlled manner involving a precisely predetermined sequence of mixing and thermal treatment or history.

In order to minimize and preferably eliminate the presence of any residual monomers it is important to ensure that the reaction is complete. This is dependent upon a number of factors such as the substrate onto which the adhesive is applied, the type and intensity of the ultra violet light and the number of ultra violet light passes.

Optional Ingredients:

Common additives known in the art such as polymerization inhibitors, chain transfer agents, surfactants, soluble or dispersible polymers, buffers, preservatives, antioxidants, pigments, mineral fillers, and the like and mixtures thereof may also be comprised within the adhesive composition in quantities up to 10% by weight each respectively. Preferably, the hydrogels herein should contain no salt or minimum levels, below 1% by wt, preferably below 0.5% by wt.

Other suitable monomers can also be incorporated at amounts up to about 50 mole % of the polymer. These monomers can be selected from e.g. strong-acid monomers: the strong acid monomer is defined in relation to its pKa, which must be below 3. The pKa is measured by titration of the acid with strong base in aqueous solution according to methods well known in the art. The said strong acid monomers are preferably selected from the group of olefinically unsaturated aliphatic or aromatic sulfonic acids such as 2-acrylamido-2-methylpropanesulfonic acid, 3-sulphopropyl (meth)acrylate, 2-sulfoethyl (meth)acrylate, vinylsulfonic acid, styrene sulfonic acid, allyl sulfonic acid, vinyl toluene sulfonic acid, methacrylic sulfonic acid and the like. Particularly preferred strong-acid monomers are 2-acrylamido-2-methyl-propanesulfonic acid, 3-sulphopropyl (meth)acrylate, 2-sulfoethyl (methacrylate; other suitable monomers can be selected from non-ionic, zwitterionic, or cationic monomers known to those skilled in the art. The non-ionic monomers are preferably hydrophilic. Hydrophilic means in this respect, that the monomer is soluble in water to an extend of at least 10 wt. %. Examples of nonionic monomers include N,N-dimethylacrylamide, acrylamide, N-isopropyl acrylamide, hydroxyethyl (meth)acrylate, hydroxypropyl (meth)acrylate, alkyl (meth)acrylates, N-vinyl pyrrolidone and the like. Examples of cationic monomers include N,N-dimethylami-noethyl (meth)acrylate, N,N-dimethylaminoethyl (meth)acrylamide and the respective quaternary salts and the like.

Residual Monomers/Impurities:

For the applications described below it is essential that the Hydrogel Adhesives show very low amount of residual starting monomers, impurities, and/or by-products that could be formed during polymerization.

The level of residual starting monomers after the said polymerization step, is preferably below 10000 ppm, preferably below 1000 ppm, more preferably below 500 ppm, even more preferably below 200 ppm, even more preferably below 100 ppm, even more preferably below 50 ppm, even more preferably below 20 ppm, and most preferably below 10 ppm.

In addition to that said hydrogels contain less than 100 ppb, preferably less than 50 ppb, and most preferably less than 20 ppb of α,β-unsaturated carbonyl by-product(s) derived from said polyol(s) during polymerization, and wherein the level of residual starting monomer(s) is below 200 ppm, preferably below 100 ppm, more preferably below 50 ppm, even more preferably below 20 ppm, and most preferably below 10 ppm.

Impurities include conjugated olefins such as acrylonitrile, acrylamide, acrolein, acrylates, t-butylacrylamide, other substituted acrylamides and the like that are introduced into the hydrogel premix in minor amounts along with the main ingredients. Some conjugated olefins can be found as impurities and also be formed as by-products of the polymerization reaction.

The by-products of the polymerization reaction refer to all products that are produced from any ingredients of the reaction medium including impurities, whatever the polymerization conditions applied are. The by-products produced from said polyol(s) are of particular concern in the present invention.

These by-products may comprise α,β-unsaturated carbonyls such as acrolein, acrylamides, acrylates, and the like. For example, as it was previously mentioned glycerol can produce acrolein as a decomposition product during the photopolymerization step. It is also known that acrylamido-2-methane propanesulfonic acid (AMPS) can decompose to generate acrylamide. Acrolein is the by-product of particular concern in the present invention. But other by-products that could derive from common additives used for making hydrogels, are within the scope of the invention.

The chemical treatment refers to any chemical reactions known in the art that may be applied to a compound. These reactions include, but are not limited to, substitution, addition, elimination, cyclisation, pericyclic reaction, oxidation, and reduction. Addition reactions are particularly preferred in the process described in the present invention.

Said treatment can be a PRE-treatment where the compound is added to the monomer solution before polymerization, e.g. directly into the solution immediately before the polymerization, or a POST-treatment where the compound is added to the polymerized hydrogel after polymerization via spraying, slot coating, printing, transfer, and the like processes in solution.

The compound that reacts with residual monomers, impurities, and/or by-products can be in particular, a nucleophile, an oxidizing agent, a reducing agent, or a conjugated diene. For the process described in the present invention, it is particularly preferred that the compound is a nucleophile.

Suitable nucleophiles include the whole range of hetero nucleophiles wherein hetero nucleophiles are nucleophiles with a polarizable heteroatom like N, S, O or P. Preferred nucleophiles are ammonia, ammonium salts of mineral and carboxylic acids (e.g. chlorides, bromides, sulfates, phosphates, formiates, acetates, acrylates, propionates, tartrates and the like), arylamines (wherein aryl preferably means monocyclic or bicyclic aromatic rings which are optionally substituted by one, two or more substituents. The substituents are independently of each other preferably selected from the group consisting of C1-C6-alkyl, OH, C1-C6-alkoxy, nitro, halogen etc. Examples are e.g. aniline, methylaniline, benzylaniline, xylidine and the like), heteroaromates (wherein heteroaromates preferably means monocyclic or bicyclic aromatic rings with one, two, or more heteroatoms like N, O, S, which are optionally substituted by one, two or more substituents. The substituents are independently of each other preferably selected from the group consisting of C1-C6-alkyl, OH, C1-C6-alkoxy, nitro, halogen etc. Preferred are N-heteroaromates. Examples are e.g. pyridine, imidazole, methylimidazole etc.), alkylamines and/or their mineral or carboxylic salts (alkylamines means preferably mono-, di- or trialkylamines with C1-C6 alkyl chains wherein two alkyl chains can form together with the N a ring of 5 or 6 members. Examples are e.g., piperidine, piperizine, mono-, di- and tri-butylamine, dimethylamine, diethylamin, dipropaneamine, triethylamine, etc.), multifunctional amines (which are preferably mono-, di- or triamines of alkyl or aryl amines. Examples are e.g. hexamethylendiamine, ethylendiamine, propanediamine diethylentriamine) polyamines (e.g. polyvinylamine), hydroxylamine, hydrazine, aminoguanidine, alkali sulfites, ammonium sulfites, alkali or ammonium hydrogen sulfites, alkali-, or ammonia-metabisulfites or -bisulfites, hydrogen halide, bromosuccinimide, pyridinium bromide, bromine, or thiols. Aminoguanidine, bisulfite and metabisulfite are particularly preferred in the present invention.

Oxidizing agents may include permanganate, bichromate, chromate, selenium dioxide, osmium tetroxide, sodium periodate, ozone, peroxides (sodium persulfate, dibenzoylperoxide etc.) or hydroperoxides (e.g. benzoylhydroperoxide, hydrogenperoxide).

Reducing agents may include metal hydrides, sodium hypochlorite, metals and their salts of mineral and carboxylic acids (e.g. chlorides, bromides, sulfates, phosphates, formiates, acetates, acrylates, propionates, tartrates and the like), Grignard reagents, alkali and ammonia sulfites, methane sulfine acids and their salts, e.g. sodium formaldehyde sulfoxylate, saccharides (e.g. ascorbic acid, glucose, frutose and the like).

Dienes may include cyclopentadiene, hexachlorocyclopentadiene, isoprene, 2-methoxybutadiene, and the like.

When the compound is a nucleophile, it is particularly preferred that it reacts with the double bond(s) of the starting monomers, impurities and/or the by-products by an addition reaction.

In the process of the present invention, the compound which reacts with said residual starting monomer(s), impurity(s) and/or by-products is preferably present in amounts of less than 30000 ppm, preferably less than 10000 ppm, more preferably less than 5000 ppm, most preferably less than 3000 ppm, with respect to the hydrogel.

Application and Use of Such Hydrogel Body Adhesives:

The possible fields of use of the described Hydrogel Adhesives are personal care products (as described for example in WO 99/00084 and WO 99/00085), health care products (as described for example in WO 97/36968 and WO 97/01311) and beauty care. In principal all applications are possible where functional articles have to be attached to the human body.

Test Methods

1. Rheology

The rheology of hydrogels is measured at 25° C. using a HAAKE RHEOS-TRESS 1 oscillatory rheometer or the equivalent. A sample of thickness of approximately 1 mm and diameter of 20 mm is placed between two insulated Parallel Plates of 20 mm diameter, controlled at a temperature of approximately 25° C. using a Peltier system or equivalent. A Dynamic Frequency Sweep is performed on the hydrogel in either stress or strain mode at an applied strain within the linear elastic response of the hydrogel (e.g., up to a strain of about 10%), with measurements at discrete frequency values between 47.75 Hz (300 rad/sec) and 0.143 Hz ( 0.8992 rad/sec). Results are quoted as G′, G″ and tan delta at frequency values of 1.0 and 100 rad/sec. The hydrogel is aged at least 24 hours before measurement. The average of at least three determinations are reported.

2. Peel Force on Dry Skin

The peel force to remove hydrogel from dry skin is measured using a suitable tensile tester, for example an Instron Model 6021, equipped with a 10N load cell and an anvil rigid plate such as the Instron accessory model A50L2R-100. Samples are cut into strips of width 25.4 mm and length between about 10 and 20 cm. A non-stretchable film of length longer than the hydrogel is applied to the reverse side of the hydrogel sample (e.g., the substrate side) using double sided adhesive. A suitable film is 23μ thick PET, available from Effegidi S.p.A, 43052, Colorno, Italy. For samples with release paper,-the release paper is removed prior to applying the hydrogel to the forearm and then rolling it into place using a compression weight roller to prevent air entrapment between hydrogel and skin. The roller is 13 cm in diameter, 4.5 cm wide and has a mass of 5 Kg. It is covered in rubber of 0.5 mm thickness. The free end of the backing film is attached to the upper clamp of the tensile tester and the arm is placed below. The sample is peeled from the skin at an angle of 90 degrees and a rate of 1000 mm/min. The average peel value obtained during peeling of the whole sample is quoted as the peel value in N/cm. The average of triplicate measurements is reported.

3. Peel Force on PET

Peel force to remove hydrogel from poly(ethylene teraphthalate) (PET) film is measured using a suitable tensile tester, for example a Zwick Z1.0/TH1S, equipped with a 50N load cell and a pneumatic grip like Zwick Model: 8195.01.00 and attachment for a rigid lower plate, e.g. steel, oriented along the direction of cross-head movement. Freshly produced hydrogel is stored in a closed aluminium bag or similar for at least 12 to 24 hours at room temperature before measuring. A defect free sample of at least 10 cm in length is cut from the hydrogel sample. A piece of double sided adhesive, for example type Duplofol 020DIVB+L from Lohmann GmbH Postoffice box 1454 56504 Neuwied, at least 130 mm long and 25.4 mm wide is stuck to the front side of the lower plate. The hydrogel is punched out with a Zwick mechanical cutting press like Zwick model 7104 using a cutting tool 25.4 mm wide and 25.4 cm long. The second liner is removed from the tape and it is stuck on the back side of the hydrogel sample. A strip of standard PET of 23μ thickness and no corona treatment, is cut to about 300 mm×28 mm. Suitable material would include “Cavilen-Forex” from Effegidi S.p.A, Via Provinciale per Sacca 55, I-43052 Colorno, Italy. The release liner is removed from the hydrogel and the bottom end fixed to the rigid plate by regular tape. The standard substrate is then applied onto the body adhesive using a hand roller once forward and once backward at a speed of 1000 to 5000 mm/min. The roller is 13 cm in diameter, 4.5 cm wide and has a mass of 5 Kg. It is covered in rubber of 0.5 mm thickness. The measurement is preferably performed within 10 minutes of application of the substrate.

The free end of the standard substrate is doubled back at an angle of 180 degrees and the rigid plate is clamped in the lower clamp of the tensile tester. The free end of the standard substrate is fixed in the upper clamp of the tensile tester. The peel test is performed at a speed of 1000 nm/min. The initial 20 mm of peel is disregarded and the average force over the remaining length is quoted as the peel force in N/cm. The average of triplicate measurements is reported.

4. pH of the Polymerized Hydrogel

The pH of the hydrogel is measured using an electronic pH meter, for example as supplied by Mettler Toledo, and a flat bulb electrode, for example type InLab 426, calibrated as per the manufacturers instructions. The bulb is brought into contact with the surface of the gel and the measurement is recorded after some seconds, once the value on the display is constant. The electrode is rinsed with distilled water between successive measurements.

5. pH of Monomer Solutions

The pH of a monomer solution can be measured using methods well known to the art. For example, an Ionlabph/ion level 2P meter can be used equipped with a SenTix 41 electrode (available from Wissenschaftlich Technische Werkstaetten).

6. Residual NaAMPS and Acrylic Acid in Polymerized Hydrogels

Sample Preparation: 100 ml of 0.9% w/v saline solution are added to 1.0000 g hydrogel and the mixture is shaken in a thermostatic bath for a minimum of 16 hours at 40° C. An aliquot of the exctract is collected into a syringe and transfered it through a 0.20 μm hydrophilic filter into a HPLC autosampler vial.

Analysis: Reversed-phase HPLC/DAD, −50 μl of the hydrogel filtrate (as above) is injected directly into the HPLC, for example an Agilent Series 1100 equipped with an Agilent Series 1100 solvent delivery module, Agilent Series 1100 auto injector, Agilent Series 1100 photo diode array detector and an Agilent Zorbax SB AQ 4.6×150 mm 5 [symbol] m analytic-column and an Agilent Zorbax SB AQ 4.6×12.5 mm as guard-coloumn. The mobile phase comprises 96% of eluent A (H₂O, containing 0.867 mmol/l Phosphoric acid) and 4% of eluent B (Acetonitrile). The flow rate is 1.2 ml/min. The analytic temperature is 30° C. A photo diode array channel 200 nm (bandwidth 5 nm) is used for detection, the UV Spectra across 190-300 nm can be applied for peak purity assessment. The level of analyte is quantified using standard procedures well known to the art and reported as micrograms analyte per gram of hydrogel (ppm). The quantitative detection limit of NaAMPS is below 5 microgram analyte per gram hyrogel (ppm). The quantitative detection limit of Acrylic Acid is below 3 microgram analyte per gram hyrogel (ppm), based on a signal/noise ratio of 10.

7. Determination of Acrolein an Acrylonitrile in Hydrogel-Samples Treated with Sodium Bisulfite

Sample Preparation:

The protective foil is removed from the “Hydrogel-Sample”. Then c. 5 g are weighed into a wide-necked bottle. To the sample 500 ml of NaCl-solution (0.9% w/w) are added. This preparation is stored at 40° C. for c. 24 hours. During normal working time the bottle is shaken vigorously every hour. After 24 hours the bottle is allowed to cool down to room temperature, then the liquid phase is separated.

Final Determination:

Principle:

Acrolein and acrylonitrile are determined via purge & trap GC-MS analysis. For purge & trap a suitable commercial autosampler can be used. The autosampler is connected to a capillary gas chromatograph coupled to a quadrupole mass spectrometer.

Off-line purge & trap can be carried out as well, then the adsorption tube has to be analysed further on a GC-MS system equipped with a thermodesorption unit.

Principle information about the analytical technique is given in EPA methods 5030B and 8260B.

For quantification an external standard procedure is recommended. Standard addition method can cause systematic errors, if residual bisulfite is present in the extract, which may react with the spiked standards. In such a case too high values are evaluated.

A portion of 5 ml (2 ml for higher concentrated or foaming sample extracts) of the separated aquatic extract is used for purge & trap GC-MS analysis.

Possible Measurement Parameters are Given Below:

For purge & trap the autosampler PTA-3000 (supplied by IMT) was used:

| | | | | | |
|---|---|---|---|---|---|
| sample temperature: | 40° | C. | | | |
| purge time: | 20 | min | purge flow: | 20 | ml He/min |
| valve temperature: | 80° | C. | transfer line: | 200° | C. |
| trap cooling | −120° | C. | water trap | −15° | C. |
| temperature: | | | temperature: | | |
| trap desorption | 200° | C. | desorption | 10 | min |
| temp.: | | | time: | | |

Chromatographic Conditions:
fused silica column:
RTX-VMS (supplied by Restec) length: 60 m, internal diameter 0.32 mm, film thickness 01.8 μm
Temp.-Progr.: 7 min isothermal at 40° C.
   40° C.-80° C. with 7 K/min
   80° C.-220° C. with 14 K/min
   13 min isothermal at 220° C.:
Injector temperature: 200° C. Transfer line temperature: 220° C.
carrier gas: helium 0.6 bar
Quadrupol MS system (e.g. MD 800 supplied by Thermo Quest)
source temperature: 220° C.:
ionisation: EI⁺
selected ion monitoring: m/z 52 and 53 for acrylonitrile (m/z 53 used for evaluation)
m/z 55 and 56 for acrolein(m/z 56 used for evaluation)

Calibration is carried out by preparing standard solutions in a NaCl-solution (0.9% w/w) at the interesting concentration level. The standard solution is analysed by purge & trap GC-MS under the same conditions like the Hydrogel extracts.

8. Determination of Acrylamide and Tert-Butyl Acrylamide in “Hydrogel-Samples

Sample Preparation:

The protective foil is removed from the “Hydrogel-Sample”. Then c. 5 g are weighed into a wide-necked bottle. To the sample 500 ml of NaCl-solution (0.9% w/w) are added. This preparation is stored at 40° C. for c. 24 hours. During normal working time the bottle is shaken vigorously every hour. After 24 hours the bottle is allowed to cool down to room temperature, then the liquid phase is separated.

Portions of 10 ml of the extract are used for further sample pretreatment, based on a bromination procedure described in EPA method 8032A.

The following procedure was carried out:

1.5 g of KBr are added, 1 drop of HBr (48% w/w in water) and 1 ml of bromine water (1.5 ml bromine/100 ml water) are added. After shaking the samples are kept for 1 h at 0° C. in an ice bath irradiation by light is avoided.

When the samples are warmed to room temperature again, 4 drops of a Na₂S₂O₃ solution (1M) are added and the samples are shaken.

Then 3 g NaCl are added and the derivatives of acrylamide and tert-butylacrylamide are extracted with 1.5 ml ethyl acetate. At this step 100 μl of an internal standard solution of 1,2-di-bromo-3-chloro propane (c. 0.04 μg/100 μl ethyl acetate) are added. The extraction is done for at least two minutes on a shaker. Then the ethyl acetate phase is separated and dried with Na₂SO₄. The dry extract is transferred into an autosampler vial where finally 3 drops of triethyl amine are added.

Final Determination:

Principle:

The derivatives of acrylamide and tert-butyl acrylamide ar determined via GC with mass selective detection in negative chemical ionization mode.

Possible Measurement Parameters are Given Below:

Chromatographic Conditions:

fused silica column:
Stabilwax-DA length: 30 m, internal diameter 0.32 mm, film thickness 0.5 μm
Temp.-Progr.: 50° C.-100° C. with 10 K/min
   100° C.-240° C. with 6 K/min
   10 min isothermal at 240° C.
Injector temperature: 250° C. Transfer line temperature: 280° C.
carrier gas: helium 0.4 bar, constant flow: 1.2 ml/min
splitless injection of 2 μl
Quadrupol MS system (e.g. HP 7973 supplied by Agilent)
source temperature: 160° C.:
ionisation: NCI with methane
selected ion monitoring: m/z 79 and 81 (m/z 79 used for evaluation)

Calibration can be done by standard addition of the analytes to aliquotes of the extracts which are prepared and analysed in the same way as the unspiked extract. Instead of standard addition an internal standard method may be used.

9. Water Activity (Relative Humidity)

Relative humidity is measured using an electronic humidity probe, for example the Testo 650 supplied by Testo GmbH & Company, calibrated as per the manufacturers instructions. A sample of hydrogel is placed inside the measuring chamber and sealed. Measurements are preferably made at approximately 25° C. The relative humidity and temperature are displayed on the instrument and recorded when constant. This is typically between about 30 minutes and several hours. The water activity is the relative humidity divided by 100.

10. Rapid Ageing Test

To simulate the conditions a consumer product has to bear during shipment and storage the samples are individually sealed in moisture-, air- and light-tight aluminum bags and stored for a specific number of days at 70° C. before they are characterized for its properties. The number of days is at least 14, preferably longer. The samples are weighed before the measurements to be sure that no waterloss has occured during storage. For the SI₁₄ and SIₓ₁₄ the sample has to be characterized after 14 days of storage at 70° C.

The yellowing is determined according to DIN 6167 with a Spectra-flash SF600V spectrometer (supplied by datacolor).

### EXAMPLES

Preparation of Na-Acrylate Solution

Na-acrylate solution is prepared by adding aqueous sodium hydroxide solution (NaOH, Aldrich, preferably 50 wt. %) to acrylic acid while keeping the temperature below 25° C. Additional water is added to adjust the solid content to 50%. The degree of neutralization is at least 50 mole %.

This aqueous solution of acrylic acid and Na-acrylate is used to prepare the pre-gel monomer mix as described below.

Preparation of Adhesive Hydrogel

### Example 1

Approximately 51.3 parts of 50 wt % Na-Acrylate (70% neutralized, preparation see above) solution, approx. 37.0 parts of glycerol and approx. 11.3 parts of deionized water are added together with approx. 0.1 to 0.3 parts crosslinker (i.e IRR 210) and approx. 0.2 parts of photoinitiator (e.g Darocure 1173 or Irgacure 2959). The procedure is carried out in brown glassware which is covered with a brown watch glass to protect the reaction mixture from light. After stirring for about 15 to 30 minutes the reaction mixture is poured on a teflon coated plate to give a 1 mm thick layer. The reaction mixture is than irradiated with a 2000 W Hönle UV at 100 mW/cm². Typical irradiation times range between 60 s to 180 s. The gels are then covered with regular photocopy paper and peeled off the plate. The other side of the gel is covered with a release liner (e.g. siliconized paper).

The peel force on PET of the hydrogel is 2.1 N/cm.

The resultant hydrogels are individually sealed in light-proof, air- and water-tight aluminum bags and stored in an oven at 70° C. The aged hydrogels are taken out of the oven after different periods of time and analyzed as described above. The samples are weighed before the measurements to be sure that no waterloss has occured during storage. The results can be seen in the following table 1.

TABLE 1

| Days | | | | | |
|---|---|---|---|---|---|
| stored at | G′ (Pa) | G″ (Pa) | tan δ | SI | |
| 70° C. | (1 rad/s) | (1 rad/s) | (1 rad/s) | index | Yellowing |
| 0 | 5714 | 2819 | 0.49 | — | 13 |
| 7 | 6240 | 3380 | 0.54 | SI₇ | 54 |
| | | | | 0.09 | |
| 14 | 5413 | 2868 | 0.53 | SI₁₄ | 59 |
| | | | | 0.05 | |
| 28 | 6097 | 3355 | 0.55 | SI₂₈ | 68 |
| | | | | 0.07 | |
| 56 | 6192 | 3178 | 0.51 | SI₅₆ | 81 |
| | | | | 0.08 | |
| 84 | 5748 | 3273 | 0.57 | SI₈₄ | 84 |
| | | | | 0.01 | |

Results:

As can be seen from the results in the above table 1 the hydrogel has been stored for a period and up to 84 days at 70° C. and the G′₂₅ (1 rad/s) is between 5714 and 6240 Pa. The SI₁₄ value is very low at 0.05. This means the gel properties do not suffer from prolonged storage at 70° C. This is essential for providing shipping and storage stable consumer products containing the said hydrogel adhesive.

### Example 2 (Comparative)

The example is done according to U.S. Pat. No. 5,665,477, tab. 2, example 3.

45.3 parts of deionized water, 65.1 parts of acrylic acid, 0.99 parts of photoinitiator (Darocure 1173), 0.51 parts of crosslinker (polyethyleneglycole 400 diacrylate), 44.7 parts of Sokalan®PA 50 (40 wt % of polyacrylic acid, partly neutralized to pH 7, molecular weight 30,000 g/mol), 3.0 parts of potassium chloride and 41.4 parts of diisopropanolamine are added together with 83.3 parts of glycerol and 15.5 parts of 50 wt % aqueous sodium hydroxide solution. The procedure is carried out as in example 1.

The peel force on PET of the hydrogel is 1.6 N/cm. On storage the prepared hydrogel gets a strong unpleasant odour.

The results of the aged samples can be seen in the following table 2:

TABLE 2

| Days stored | G′ (Pa) | G″ (Pa) | tan δ | SI | |
|---|---|---|---|---|---|
| at 70° C. | (1 rad/s) | (1 rad/s) | (1 rad/s) | index | Yellowing |
| 0 | 6009 | 2717 | 0.45 | — | 13 |
| 7 | 5283 | 2816 | 0.53 | SI₇ | 65 |
| | | | | 0.12 | |
| 14 | 7104 | 3953 | 0.56 | SI₁₄ | 84 |
| | | | | 0.18 | |

Results:

As can be seen from the results in the above table 2 the SI₁₄ value and the degree of discolouration are quite higher than in example 1.

### Example 3

### Post-Treatment with Sodium Bisulfite

Gels made according to example 1 are post treated with 3000 ppm sodium bisulfite by spraying an aqueous solution of sodium bisulfite to the polymerized gel before covering it with the release liner. The analysis for residual monomer, impurities or by-products is performed after 24 hours (see table 3)

TABLE 3

| | Acrylic Acid | Acrolein |
|---|---|---|
| Hydrogel treated with | (ppm) | (ppm) |
| 0 ppm NaHSO₃ | 1166 | 0.03 |
| 3000 ppm NaHSO₃ | 25 | <0.01 |

### Example 4 (Comparative Formulation)

Approximately 63.8 parts of 50 wt % Na-Acrylate (10% neutralized, 50 wt. % solid content) solution, approx. 33.9 parts of glycerol and approx. 0.02 parts of deionized water are added together with approx. 0.1 to 0.3 parts crosslinker (i.e IRR 210) and approx. 0.2 parts of photoinitiator (e.g Darocure 1173 or Irgacure 2959). The procedure is carried out in brown glassware which is covered with a brown watch glass to protect the reaction mixture from light. After stirring for about 15 to 30 minutes the reaction mixture is poured on a teflon coated plate to give a 1 mm thick layer. The reaction mixture is than irradiated with a 2000 W Höbnle UV lamp at 100 mW/cm². Typical irradiation times range between 60 s to 180 s. The gels are then covered with regular photocopy paper and peeled off the plate. The other side of the gel is covered with a release liner (e.g. siliconized paper).

The resultant hydrogels are individually sealed in light-proof, air- and water-tight aluminum bags and stored in an oven at 70° C. The aged hydrogels are taken out of the oven after different periods of time and analyzed as described above. The samples are weighed before the measurements to be sure that no waterloss has occured during storage. The results can be seen in the following table 4:

TABLE 4

| Days | | | | |
|---|---|---|---|---|
| stored at | G′ (Pa) | G″ (Pa) | tan δ | SI |
| 70° C. | (1 rad/s) | (1 rad/s) | (1 rad/s) | index |
| 0 | 16675 | 6326 | 0.38 | — |
| 7 | 24555 | 4497 | 0.18 | SI₇ |
| | | | | 0.47 |
| 14 | 41935 | 2142 | 0.05 | SI₁₄ |
| | | | | 1.51 |
| 34 | 102235 | 5328 | 0.05 | SI₃₄ |
| | | | | 5.13 |

As can be seen from the results in the above table 4 the hydrogel has been stored for only 34 days at 70° C. and the G′₂₅ (1 rad/s) has gone up from 16675 Pa to 102235. The SI₁₄ value is 1.51 and the SI₃₄ value is 6.13. This means the gel properties do severely suffer from prolonged storage at 70° C.

### Example 5:

In analogy to the examples 1-3, which describe a batch production of the Hydrogel in the lab scale the process can also be carried out continuously in a pilot line or production line. The compositions of the monomer mix are unchanged compared to the laboratory samples. The preparation of the monomer mix takes place in a stirred tank reactor or the like. The monomer mixture is extruded onto a substrate (e.g a nonwoven webbing) at a basis weight of approximately 1.0 kilograms per square meter. Polymerization is carried out by irradiating with UV light using 1 to 7 2000 W Hönle UV lamps or 1 to 12 high power IST UV lamps or a combination of both. The lamps can be equipped with glass filters that cut wavelength below 320 nm. By this process the monomer solution is converted into an adhesive hydrogel. After passing the UV lamps this adhesive hydrogel is covered with a release liner (e.g siliconized paper or oriented polypropylene (OPP) foil), trimmed to the required width and wound up onto rolls. Instead of rolls any other form, e.g. festooning, for storage of continuous material is imaginable.

1. A hydrogel adhesive comprising 10-60 wt % of a cross-linked hydrophilic polymer, 5-80% of a water-soluble non-ionic humectant, and from about 10-85 wt % water, wherein the hydrophilic polymer is prepared by polymerizing a mixture which comprises at least 80 mole % of one or more weak-acid monomer units having a pKa above 3, the weak-acid monomer being more than 60 mole % in its salt form, the level of monomer in acid form in said hydrophilic polymer not exceeding 50 mole % of all monomer units and the hydrogel adhesive having a peel strength on PET of 0.3 to 5.0 N/cm and a stability index measured after 14 days SIₓ₁₄ below 0.50.

2. The hydrogel adhesive of claim 1 with having a stability index measured after 14 days SIₓ₁₄ below 0.10.

3. The hydrogel adhesive of claim 1 wherein the hydrogel adhesive is free of an alkanolamine.

4. The hydrogel adhesive of claim 1 wherein the weak-acid monomer is selected from the group consisting of acrylic acid and methacrylic acid.

5. The hydrogel adhesive of claim 1 wherein the weak-acid monomer is present from 60 mole % to 80 mole %, in its salt form.

6. The hydrogel adhesive of claim 1 wherein said water-soluble nonionic humectant comprises a polyhydric alcohol.

7. The hydrogel adhesive of claim 1 wherein the hydrophilic polymer comprises at least 90 mole % weak-acid monomer units.

8. The hydrogel adhesive of claim 1 having a pH value of 4.0 to 8.0.

9. The hydrogel adhesive of claim 1 wherein the water-soluble non-ionic humectant is glycerol and the weak acid is acrylic acid.

10. The hydrogel adhesive of claim 1 wherein the counterion for the acrylic acid unit in salt form is a mono, di, or tri-valent metal ion or a combination thereof.

11. A hydrogel adhesive having a stability index measured after 14 days SI₁₄ below 0.10.

12. A hydrogel adhesive having a stability index measured after 14 days SIₓ₁₄ below 0.10.

13. The hydrogel adhesive of claim 11 wherein the hydrogel adhesive has a peel strength on PET of 0.3 to 3.0 N/cm.

14. The hydrogel adhesive of claim 1 having a G′₂₅ (1 rad/sec) in the range 100 to 20000 Pa.

15. The hydrogel adhesive of claim 1 wherein the residual monomer concentration in the hydrogel adhesive is below 10000 ppm.

16. The hydrogel adhesive of claim 1 containing less than 100 ppb of an α,β-unsaturated carbonyl by-product derived from said polyol during polymerization, and wherein the level of residual starting monomer is below 200 ppm.

17. The hydrogel adhesive of claim 1 wherein the low levels of residual monomers, impurities and by-products is achieved by pretreating or posttreating with a compound that is capable of reacting with said residual monomers, impurities and byproducts.

18. The hydrogel adhesive of claim 17 wherein the compound capable of reacting with the residual monomers, impurities, and by-products is a nucleophile.

19. The hydrogel adhesive of claim 17 wherein the compound is sodium bisulfite.

20. (canceled)

21. The hydrogel adhesive of claim 1 wherein the weak-acid monomer comprises acrylic acid.

22. The hydrogel adhesive of claim 1 wherein water-soluble nonionic humectant comprises glycerol.

23. The hydrogel adhesive of claim 12 wherein the hydrogel adhesive has a peel strength on PET of 0.3 to 3.0 N/cm.

24. A method of attaching a functional article to mammalian skin comprising applying a hydrogel adhesive of claim 1 to a surface of the functional article, and contacting mammalian skin to the surface of the functional article having the hydrogel adhesive applied thereto.
